(19) United States
(12) Patent Application Publication
(10) Pub. No.: US 2011/0190479 A1
(43) Pub. Date: Aug. 4, 2011

(54) DIFFERENTIAL TARGETING OF TRIACYLGLYCEROL SYNTHASE AND WAX SYNTHASE ACTIVITY OF THE TRIACYLGLYCEROL SYNTHASE GENE FOR TUBERCULOSIS TREATMENT

(76) Inventors: PAPPACHAN E. KOLATTUKUDY, TITUSVILLE, FL (US); TATIANA SIRAKOVA, ORLANDO, FL (US); VINOD S. DUBEY, WAUKEGAN, IL (US); JAIYANTH DANIEL, ORLANDO, FL (US)

(21) Appl. No.: 12/983,704

(22) Filed: Jan. 3, 2011

Related U.S. Application Data
(63) Continuation-in-part of application No. 12/546,368, filed on Aug. 24, 2009.
Application No. 12/546,368 is a continuation of application No. 11/561,477, filed on Nov. 20, 2006, now Pat. No. 7,579,012.
(60) Provisional application No. 60/748,283, filed on Dec. 7, 2005.

(51) Int. Cl. C07K 16/12; C12Q 1/48; C07H 21/04

(52) U.S. Cl. 530/389.5; 435/15; 536/24.5

(57) ABSTRACT

The application reports the discovery that mycobacterial triacylglycerol synthase (MTTGS) enzymes also have a wax synthase function. This bifunctional enzymatic activity, or ‘dual function,’ implies that the enzymes contain separate binding sites for diacylglycerol and acyl alcohol and a common binding site for acyl-coenzyme A. Embodiments of the invention relate to methods of selection and/or design of therapeutic compounds that will reduce any potentially toxic side effects of candidate drugs designed to inhibit the mycobacterial enzyme.

### CROSS-REFERENCE TO RELATED APPLICATIONS

This application is a continuation in part of U.S. patent application Ser. No. 12/546,368, filed Aug. 24, 2009, which is a continuation of U.S. patent application Ser. No. 11/561,477; filed Nov. 20, 2006, now U.S. Pat. No. 7,579,012, which is related to U.S. Provisional Application No. 60/748,283 filed Dec. 7, 2005. This application is also related to U.S. Provisional Application No. 61/291,906 filed Jan. 3, 2010. Priority is claimed to foregoing under 35 USC §§119 & 120.

### GOVERNMENT SUPPORT

This invention was made through support from the NIH, Grant Nos. A146582 and A135272. The government has certain rights in this invention.

### BACKGROUND

Tuberculosis (TB) is a wide-spread disease acquired through the respiratory route. Infected individuals spread this infection efficiently by coughing or sneezing “droplet nuclei” which contain viable bacilli. Tuberculosis has been a major health problem for most of recorded history and Mycobacterium tuberculosis remains one of the world's most significant pathogens. It is the leading cause of death from a single infectious agent, and is responsible for millions of new cases of tuberculosis annually. (see e.g. Pablo-Mendez et al., (1998) New Engl. J. Med. 338, 1641-1649). Due to the airborne nature of the infection, overcrowded living conditions and shared air space are especially conducive to the spread of TB. As a result, there have been increases in infection rates of TB observed in the U.S. in prison inmates and among the homeless in larger cities.

The emergence of multi-drug resistant strains of Mycobacterium tuberculosis poses serious threats to the control of the disease due to the complex nature of second-line drug treatment (WHO Report. (2004) WHO/HTM/TB/2004.343). Upon infection, the bacterium goes through an initial replicative phase inside the alveolar macrophages after which it enters a non-replicative, drug-resistant state of dormancy. This state of dormancy is probably induced by the environmental stress exerted upon the pathogen by the host's immune response. The bacterium is able to survive in this dormant state for decades until the host's immune system is weakened when it reactivates and causes the infectious disease (Dannenberg, Jr., A. M., and Rook G. A. W. (1994) In Tuberculosis: Pathogenesis, Protection and Control, Bloom, B. R., (Ed.) American Society of Microbiology, Washington D.C.). The current anti-mycobacterial drugs are able to kill only the actively replicating mycobacteria and do not clear the latent bacteria from the host (Honer zu Bentrup, K., and Russell D. G. (2001) Trends Microbiol. 9, 597-605). Thus, latency is a major problem in TB control. One-third of the world population is infected with the latent microorganism and nearly two million deaths occur annually (Dye, C., Scheele, S., Dolin, P., Pathania, V., and Raviglione M. C. (1999) JAMA. 282, 677-686, WHO Report. (2005) WHO/HTM/TB/2005). Individuals carrying a latent infection are estimated to harbor a 2-23% lifetime risk of reactivation (Zahrt, T. C. (2003). Microbes Infect. 5, 159-167).

### BRIEF DESCRIPTION OF THE DRAWINGS

Table 1: Mycobacterium tuberculosis TGS1 (Rv3130c) is a dual-function triacylglycerol synthase/wax synthase. Table 1 shows a comparison of the TGS activity and WS activity of three triacylglycerol synthase (TGS) genes, Rv3130c (MTTGS1), Rv3734c (MTTGS2), and Rv3740c. Triacylglycerol synthase (TGS) genes were expressed in E. coli and total cell lysates were assayed for TGS and wax synthase (WS) activities. The TGS assays were performed with lysates containing 150 μg protein in 0.1 M citrate/phosphate buffer, pH 6.5, containing 1 mg BSA, 10 mM MgCl₂, 500 μM [¹⁴C]diolein (specific activity 1.6 Ci mol⁻¹), 100 μM oleyl (C_{18:1}) alcohol as substrates. Following the assay, the reaction mixtures were extracted and the TG or WS product was quantified as described previously (Daniel et al., 2004). nd, not determined. Rv3130c (TGS1) showed by far the highest TGS and WS activities of the three genes. The mycobacterial TGS1 appears to be a genuine bifunctional enzyme with comparable TGS and WS activities.

### DETAILED DESCRIPTION

The ability of Mycobacterium tuberculosis to go into a latent/dormant state and survive under such conditions for decades makes TB control extremely difficult. One way to fight against latent TB is to develop drugs to target the ability of the pathogen to survive under such latent conditions for extended periods of time.

The inventors have discovered that certain TB genes (e.g., Rv 3130c) encode enzymes required for Mycobacterium tuberculosis to store energy in order to enter and survive the dormancy (or latent) period. In conjunction, the inventors have also found that triacylglycerol (TG) can be used as an energy source by M. tuberculosis during the dormancy period. Thus, its synthesis could be an ideal drug target against latent TB.

At least two enzymes, DGAT1 and DGAT2, belonging to two different gene families, play critical roles in the biosynthesis of triacylglycerol (TG) in humans. It has been shown that DGAT1 catalyzes the formation of TG and waxes and DGAT2 shows only TGS activity but not the WS activity in vitro. (Yen C L, Monetti M, Biurri B J, Farese R V Jr. (2005) The triacylglycerol synthesis enzyme DGAT1 also catalyzes the synthesis of diacylglycerols, waxes, and retinyl esters. J Lipid Res. 46:1502-1511.) Mice lacking DGAT1 are viable and showed about 50% reduction in TG levels, but DGAT2-lacking mice showed nearly 90% reduction in TG content and were not viable after birth due to dehydration resulting from the loss of barrier function of the skin. (Smith S J, Cases S, Jensen D R, Chen H C, Sande E, Tow B, Sanan D A, Raber J, Eckel R H, Farese R V Jr. (2000) Obesity resistance and multiple mechanisms of triglyceride synthesis in mice lacking Dgat. Nat. Genet. 25:87-90; Stone S J, Myers H M, Watkins S M, Brown B E, Feingold K R, Elias P M, Farese R V Jr. (2004) Lipopenia and skin barrier abnormalities in DGAT2-deficient mice. J Biol. Chem. 279:11767-11776.) The mycobacterial TGSs do not appear to be related to the human DGATs based on inventors analysis of the amino acid sequences. Thus, the inventors have realized that the off-target effects of mycobacterial TGS-specific inhibitors should be low.

Moreover, the inventors have identified a bifunctional enzymatic activity of mycobacterial triacylglycerol synthase (MTTGS). This bifunctional enzymatic activity, or ‘dual function,’ implies that the enzymes contain separate binding sites for diacylglycerol and acyl alcohol and a common binding site for acyl-coenzyme A. This unique characteristic of mycobacterial discovered by inventors offers a rational basis for designing inhibitors that are specific for the mycobacterial enzyme and do not affect human synthase. Molecules that mimic the structural characteristics of acyl alcohols could be designed to bind to the acyl alcohol binding pocket of the mycobacterial synthase and avoid binding to human DGATs. Inventors have realized that such specific targeting enables the selection and/or design of therapeutic compounds that will reduce any potentially toxic side effects of candidate drugs designed to inhibit the mycobacterial enzyme.

Due to the dual functional nature of mycobacterial TGS discovered by inventors, it is noted that novel inhibitors can be designed to specifically target the mycobacterial enzyme and avoid inhibition of the human DGATs thereby reducing toxic side effects that may arise from such candidate drugs.

The triacylglycerol synthases in Mycobacterium tuberculosis are homologous to the bifunctional wax synthase/diacylglycerol acyltransferase (WS/DGAT) of Acinetobacter calcoaceticus. Kalscheuer R, Steinbuchel A. (2003) A novel bifunctional wax ester synthase/acyl-CoA:diacylglycerol acyltransferase mediates wax ester and triacylglycerol biosynthesis in Acinetobacter calcoaceticus ADP1. J Biol. Chem. 278:8075-8082. Based on the inventors earlier findings, Rv3130c and Rv3734c showed the highest TGS activity while Rv3734c and Rv3740c showed the highest WS activities. The three gene products were tested for their dual TGS/WS activities using physiologically relevant substrates.

The gene sequence of Rv3130c is provided as SEQ ID NO: 1. The inventors have discovered that triacylglycerol (TG) can be used as an energy source by M. tuberculosis during the dormancy period, thus its synthesis could be an ideal drug target against latent TB. SEQ ID NO: 2 shows one example of a M. tuberculosis TG synthase (MTTGS) polypeptide. Polypeptides useful in accordance with the teachings herein are further described herein. MUGS polynucleotides and polypeptides as described herein may be implemented to identify compounds to treat TB. In a preferred embodiment, a MTTGS polypeptide is one comprising at least 90-98 percent identity with SEQ ID NO. 2 and which also comprises an active site HHCMADG (SEQ ID NO. 3).

As discussed below, certain embodiments of the invention pertain to agents that bind and/or modulate MTTGS polypeptides. Agents can be designed which are specific to SEQ ID NO. 2, or a fragment of at least 5 amino acids thereof. In another embodiment, agents are designed so as to bind to SEQ ID NO. 3.

Triacylglycerol synthase (TGS) genes were expressed in E. coli and total cell lysates were assayed for TGS and wax synthase (WS) activities. The TGS assays were performed with lysates containing 150 μg protein in 0.1 M citrate/phosphate buffer, pH 6.5, containing 1 mg BSA, 10 mM MgCl₂, 500 μM [¹⁴C]diolein (specific activity 1.6 Ci mol⁻¹), 100 μM oleyl (C_{18:1}) alcohol as substrates. Following the assay, the reaction mixtures were extracted and the TG or WS product was quantified as described previously (Daniel et al., 2004). nd, not determined.

As shown in Table 1, inventors have found that Rv3130c (TGS1) showed by far the highest TGS and WS activities when compared with Rv3734c and Rv3740c. The mycobacterial TGS1 therefore appears to be a genuine bifunctional enzyme with comparable TGS and WS activities.

Thus, according to one embodiment, the invention pertains to a method for screening for therapeutic agents useful in the treatment of Mycobacterium tuberculosis infection in a mammal comprising the steps of i) identifying a molecule structurally designed to bind to a mycobacterial enzyme, ii) contacting the molecule to the mycobacterial enzyme so as to structurally bind specifically to the mycobacterial enzyme, wherein the molecule is designed to inhibit the mycobacterial enzyme.

In another embodiment, the invention pertains to a method for selectively identifying inhibitors potentially useful in the treatment of Mycobacterium tuberculosis infection in a mammal comprising the steps of i) identifying a Mycobacterium tuberculosis triacylglycerol synthase (MTTGS) enzyme producing triacylglycerol synthase (TGS) and wax synthase (WS) activity, ii) targeting the MTTGS with a molecule, wherein the molecule is structurally designed to specifically contact and bind to the MUGS producing both TGS and WS, wherein the molecule is designed to inhibit the mycobacterial enzyme, decreasing the activity of Mycobacterium tuberculosis triacylglycerol synthase.

Thus, according to one embodiment, the invention pertains to a method of screening for therapeutic agents useful in the treatment of Mycobacterium tuberculosis infection in a mammal comprising the steps of i) contacting a test compound with a MTTGS polypeptide, ii) detecting binding of said test compound to said MTTGS polypeptide.

Another embodiment of the subject invention pertains to a method of screening for agents potentially useful in the treatment of Mycobacterium tuberculosis infection in a mammal comprising the steps of i) determining the wax synthase activity of a MTTGS polypeptide sequence in the presence of a test compound or in the absence of said test compound, and identifying a test compound that whose presence modulates the wax synthase activity of said MTTGS polypeptide as a potential therapeutic agent for decreasing the activity of Mycobacterium tuberculosis triacylglycerol synthase.

According to another embodiment, the subject invention pertains to a method of screening for agents potentially useful in the treatment of Mycobacterium tuberculosis infection in a mammal comprising the steps of i) determining the wax synthase and triacylglycerol synthase activity of a MTTGS polypeptide sequence in the presence of a test compound or in the absence of said test compound, and identifying a test compound that whose presence modulates the wax synthase activity more than the triacylglycerol synthase activity of said MTTGS polypeptide as a potential therapeutic agent for decreasing the activity of Mycobacterium tuberculosis triacylglycerol synthase.

### MTTGS Polynucleotides, Polypeptides and Variants

Aspects of the invention therefore include polynucleotides encoding at least one MTTGS polypeptide and amino acid sequences representing at least one MTTGS polypeptide. Aspects of the invention also include subunits or variants of polynucleotides or MTTGS polypeptides encoded by those polynucleotides.

It is well known in the art that a single amino acid may be encoded by more than one nucleotide codon—and that the nucleotide sequence may be easily modified to produce an alternate nucleotide sequence that encodes the same peptide. Therefore, alternate embodiments of the present invention include alternate DNA sequences encoding peptides containing the amino acid sequences described for an MTTGS polypeptide. DNA sequences encoding peptides containing the claimed amino acid sequence include DNA sequences which encode any combination of the claimed sequence and any other amino acids located N-terminal or C-terminal to the claimed amino acid sequence.

It is to be understood that amino acid and nucleic acid sequences may include additional residues, particularly N- or C-terminal amino acids or 5′ or 3′ nucleotide sequences, and still be essentially as set forth in the sequences disclosed herein, as long as the sequence produces a functionally similar polypeptide or protein. A nucleic acid fragment of almost any length may be employed, and may be combined with other DNA sequences, such as promoters, polyadenylation signals, additional restriction enzyme sites, multiple cloning sites, other coding segments, and the like. Therefore, overall length may vary considerably.

MTTGS polypeptides, as used herein, may comprise short fragments of proteins often referred to as peptides, as well as longer fragments generally referred to as polypeptides, and full-length proteins. These polypeptides can be prepared by standard peptide synthesis methods known to those of skill in the art, but may also be produced using an expression vector having a polynucleotide sequence encoding the polypeptide(s) of choice operably linked to appropriate promoter, terminator, and other functional sequences (such as a sequence encoding a purification tag) to facilitate expression and purification of the peptides.

The term “moderately stringent conditions”, as used herein, means conditions in which non-specific hybridization will not generally occur. Hybridization under such conditions can be performed based on the description provided in Molecular Cloning: A Laboratory Manual 2nd ed., published by cold Spring Harbor Laboratory in 1989, edited by T. Maniatis et al. Typically, for stringent hybridization conditions a combination of temperature and salt concentration should be chosen that is approximately 12-20° C. below the calculated Tₘ of the hybrid under study. The Tₘ of a hybrid between a polynucleotide of interest or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C. According to another example, stringent conditions include incubation with a probe in 6×SSC containing 0.5% SDS, 5×Denhardt's solution and 100 micrograms/ml salmon sperm DNA at 60 degrees C.

Additional nucleic acid bases may be added either 5′ or 3′ to the MTTGS polynucleotides, and may be combined with other DNA sequences, such as promoters, polyadenylation signals, additional restriction enzyme sites, multiple cloning sites, other coding segments, and the like. Therefore, overall length of such a polynucleotide may vary considerably. In a method described by the present invention, a nucleotide sequence of SEQ ID NO: 1 is inserted into a protein expression vector to produce a protein which can be used to synthesize a DNA copy of an RNA molecule. The DNA can then be amplified to form multiple copies.

“Control sequences” are those DNA sequences that are necessary for the expression of a protein from a polynucleotide sequence containing such a sequence, operably linked to the polynucleotide sequence encoding the protein. These sequences include prokaryotic sequences such as, for example, promoters, operators, and ribosome binding sites, and eukaryotic sequences such as, for example, promoters, enhancers, and polyadenylation signals. “Expression systems” are DNA sequences (such as, for example, plasmids) appropriate for expression of a target protein in a particular host cell, these sequences comprising appropriate control sequences for protein expression in the host cell operably linked to the polynucleotide sequence encoding the target protein.

It is to be understood that a “variant” of a polypeptide is not completely identical to the native protein. A variant MTTGS polypeptide, for example, can be obtained by altering the amino acid sequence by insertion, deletion or substitution of one or more amino acids. The amino acid sequence of the protein can be modified, for example, by substitution to create a polypeptide having substantially the same or improved qualities as compared to the native polypeptide. The substitution may be a conserved substitution. A “conserved substitution” is a substitution of an amino acid with another amino acid having a side chain that is similar in polar/nonpolar nature, charge, or size. The 20 essential amino acids can be grouped as those having nonpolar side chains (alanine, valine, leucine, isoleucine, proline, phenylalanine, and tryptophan), uncharged polar side chains (methionine, glycine, serine, threonine, cystine, tyrosine, asparagine and glutamine), acidic side chains (aspartate and glutamate), and basic side chains (lysine, arginine, and histidine). Conserved substitutions might include, for example, Asp to Glu, Asn, or Gln; H is to Lys, Arg or Phe; Asn to Gln, Asp or Glu; and Ser to Cys, Thr or Gly. Alanine, for example, is often used to make conserved substitutions.

To those of skill in the art, variant polypeptides can be obtained by substituting a first amino acid for a second amino acid at one or more positions in the polypeptide structure in order to affect biological activity. Amino acid substitutions may, for example, induce conformational changes in a polypeptide that result in increased biological activity.

Those of skill in the art may also make substitutions in the amino acid sequence based on the hydrophilicity index or hydropathic index of the amino acids. A variant amino acid molecule of the present invention, therefore, has less than one hundred percent, but at least about fifty percent, and preferably at least about eighty to about ninety-eight percent amino acid sequence homology or identity to the amino acid sequence of a polypeptide comprising SEQ ID NO: 2, or a polypeptide encoded by SEQ ID NO: 3. Therefore, the amino acid sequence of the variant MTTGS corresponds essentially to the native MTTGS polypeptide amino acid sequence. A variant may also be a truncated “fragment,” as compared to the corresponding protein comprising SEQ ID NO 2, the fragment being only a portion of the full-length protein. One example of a fragment is SEQ ID NO. 3, which pertains to the active motif of SEQ ID NO. 2.

In some embodiments, the MTTGS polypeptide variants may comprise SEQ ID NO. 2 with conserved substitutions at any position other than the active motif (SEQ ID NO. 3). In other embodiments, variants according to the invention will comprise 1-5 conserved substitutions in at least one amino acid position of a fragment of SEQ ID NO. 2, wherein the fragment comprises the active motif (SEQ ID NO:3).

### Screening Methods

The invention provides assays for screening test compounds which bind to or modulate the wax synthase and/or triacyl glycerol activity of an MTTGS polypeptide or bind to and inhibit or affect expression of an MTTGS polynucleotide. A test compound preferably binds to an MTTGS polypeptide. In a more specific embodiment, a test compound decreases or increases wax synthase activity of MTTGS with little or no affect on triacylglycerol activity. In an even more specific embodiment, the test compound modulates wax synthase activity by at least about 10, preferably about 50, more preferably about 75, 90, or 100% relative to the absence of the test compound.

### 1.1. Test Compounds

Test compounds relate to agents that potentially have therapeutic activity, i.e., bind to or modulate the wax synthase and/or triacyl glycerol activity of an MTTGS polypeptide or bind to or affect expression of an MTTGS polynucleotide. Alternatively, test compounds modulate differentially the waxy synthase activity relative to the triacyl glycerol activity. Preferably, the test compounds modulate the wax synthase activity with generally no modulation of triacylglycerol activity. Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994).

### 1.2. High Throughput Screening

Test compounds can be screened for the ability to bind to and inhibit MTTGS polypeptides or polynucleotides or to affect MTTGS activity or MTTGS gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format. Alternatively, “free format assays,” or assays that have no physical barrier between samples, can be used.

### 1.3. Binding Assays

For binding assays, the test compound is preferably, but not necessarily, a small molecule which binds to and occupies, for example, the active site of the MTTGS polypeptide, such that normal biological activity is prevented. Examples of such small molecules include, but are not limited to, small peptides or peptide-like molecules.

In binding assays, either the test compound or the MTTGS polypeptide can comprise a detectable label, such as a fluorescent, radioisotopic, chemiluminescent, or enzymatic label, such as horseradish peroxidase, alkaline phosphatase, or luciferase. Detection of a test compound which is bound to the MTTGS polypeptide can then be accomplished, for example, by direct counting of radioemmission, by scintillation counting, or by determining conversion of an appropriate substrate to a detectable product.

Those skilled in the art equipped with teachings herein will appreciate that there are multiple conventional methods of detecting binding of a test compound. For example, binding of a test compound to a MTTGS polypeptide can be determined without labeling either of the interactants. A microphysiometer can be used to detect binding of a test compound with an MTTGS polypeptide. A microphysiometer (e.g., CYTOSENSOR™) is an analytical instrument that measures the rate at which a cell acidifies its environment using a light-addressable potentiometric sensor (LAPS). Changes in this acidification rate can be used as an indicator of the interaction between a test compound and an MTTGS polypeptide (McConnell et al., Science 257, 19061912, 1992).

In another alternative example, determining the ability of a test compound to bind to an MTTGS polypeptide can be accomplished using a technology such as real-time Bimolecular Interaction Analysis (BIA) (Sjolander & Urbaniczky, Anal Chem. 63, 23382345, 1991, and Szabo et al., Curr. Opin. Struct. Biol. 5, 699705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, an MTTGS polypeptide can be used as a “bait protein” in a two-hybrid assay or three-hybrid assay (see, e.g., U.S. Pat. No. 5,283,317; Zervos et al., Cell 72, 223232, 1993; Madura et al., J. Biol. Chem. 268, 1204612054, 1993; Bartel et al., BioTechniques 14, 920924, 1993; Iwabuchi et al., Oncogene 8, 16931696, 1993; and Brent WO94/10300), to identify other proteins which bind to or interact with the MTTGS polypeptide and modulate its activity.

In many screening embodiments, it may be desirable to immobilize either the MTTGS polypeptide (or polynucleotide) or the test compound to facilitate separation of bound from unbound forms of one or both of the interactants, as well as to accommodate automation of the assay. Thus, either the MTTGS polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the MTTGS polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a MTTGS polypeptide (or polynucleotide) can be accomplished in any vessel suitable for containing the reactants. Examples of such vessels include microtiter plates, test tubes, and microcentrifuge tubes.

In a specific embodiment, the MTTGS polypeptide may be a fusion protein comprising a domain that allows the MTTGS polypeptide to be bound to a solid support. For example, glutathione S-transferase fusion proteins can be adsorbed onto glutathione sepharose beads (Sigma Chemical, St. Louis, Mo.) or glutathione derivatized microtiter plates, which are then combined with the test compound or the test compound and the nonadsorbed MTTGS polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a MTTGS polypeptide (or polynucleotide) or a test compound can be immobilized utilizing conjugation of biotin and streptavidin. Biotinylated MTTGS polypeptides (or polynucleotides) or test compounds can be prepared from biotinNHS(Nhydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a MTTGS polypeptide, polynucleotide, or a test compound, but which do not interfere with a desired binding site, such as the active site of the MTTGS polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the MTTGS polypeptide or test compound, enzyme-linked assays which rely on detecting an activity of the MTTGS polypeptide, and SDS gel electrophoresis under non-reducing conditions.

Screening for test compounds which bind to a MTTGS polypeptide or polynucleotide also can be carried out in an intact cell. Any cell which comprises a MTTGS polypeptide or polynucleotide can be used in a cell-based assay system. A MTTGS polynucleotide can be naturally occurring in the cell or can be introduced using techniques such as those described above. Binding of the test compound to a MTTGS polypeptide or polynucleotide is determined as described above.

### 1.4. Enzyme Assays

Test compounds can be tested for the ability to increase or decrease the TGS or WS activity of a MTTGS polypeptide. TGS or WS activity can be measured, for example, by adapting techniques such as that described in U.S. Pat. No. 4,529,693. Enzyme assays can be carried out after contacting either a purified MTTGS polypeptide, a cell membrane preparation, or an intact cell with a test compound. A test compound which differentially modulates WS activity relative to TGS activity of a MTTGS polypeptide by at least about 10, preferably about 50, more preferably about 75, 90, or 100% is identified as a potential therapeutic agent for decreasing MTTGS activity. A test compound which increases TGS MTTGS polypeptide by at least about 10, preferably about 50, more preferably about 75, 90, or 100% is identified as a potential therapeutic agent for increasing TGS activity.

### 1.5. Gene Expression

In another embodiment, test compounds which increase or decrease MTTGS gene expression are identified. An MTTGS polynucleotide is contacted with a test compound, and the expression of an RNA or polypeptide product of the MTTGS polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of MTTGS mRNA or polypeptide expression in the cells can be determined by methods well known in the art for detecting mRNA or polypeptide. Either qualitative or quantitative methods can be used. The presence of polypeptide products of an MTTGS polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a MTTGS polypeptide.

Such screening can be carried out either in a cell-free assay system or in an intact cell. Any cell which expresses a MTTGS polynucleotide can be used in a cell-based assay system. The MTTGS polynucleotide can be naturally occurring in the cell or can be introduced using techniques such as those described above. Either a primary culture or an established cell line, such as CHO or human embryonic kidney 293 cells, can be used.

### 2. Pharmaceutical Compositions

The invention also pertains to pharmaceutical compositions comprising one or more therapeutic agents that are identified by screening methods that utilize MUGS polypeptides and/or polynucleotides. Therapeutic agent(s) can be administered to a patient to achieve a therapeutic effect, i.e. useful in treatment of TB. Pharmaceutical compositions of the invention can comprise, for example, therapeutic agents identified by a screening method embodiment described herein, which are identified by their ability to bind to or affect activity of MTTGS polypeptides, or bind to and/or affect expression MTTGS polynucleotides. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

This invention further pertains to the use of novel agents identified by the screening assays described above. Accordingly, it is within the scope of this invention to use a therapeutic agent identified as described herein in an appropriate animal model. For example, an agent identified as described herein (for example, but not limited to, a modulating agent, an antisense nucleic acid molecule, a specific antibody, ribozyme, or a MTTGS polypeptide binding molecule) can be used in an animal model to determine the efficacy, toxicity, or side effects of treatment with such an agent. Furthermore, this invention pertains to uses of novel agents identified by the above described screening assays for treatments as described herein.

Those skilled in the art will appreciate that numerous delivery mechanisms are available for delivering a therapeutic agent to an area of need. By way of example, the agent may be delivered using a liposome as the delivery vehicle. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 106 cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

2.1 Inhibitor Agents

An antisense nucleic acid molecule may be single or double stranded (e.g. a small interfering RNA (siRNA)), and may function in an enzyme-dependent manner or by steric blocking. Antisense molecules that function in an enzyme-dependent manner include forms dependent on RNase H activity to degrade target mRNA. These include single-stranded DNA, RNA and phosphorothioate molecules, as well as the double-stranded RNAi/siRNA system that involves target mRNA recognition through sense-antisense strand pairing followed by degradation of the target mRNA or by the RNA-induced silencing complex. Steric blocking antisense, which are RNase-H independent, interferes with gene expression or other mRNA-dependent cellular processes by binding to a target mRNA and getting in the way of other processes. Steric blocking antisense includes 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce MTTGS translation such that the level of MTTGS polypeptide is reduced. siRNAs mediate post-transcriptional gene silencing in a sequence-specific manner. See, for example, see website bearing the address: ambion.com/techlib/hottopics/rnai/rnai_may2002_print (last retrieved May 10, 2006). Once incorporated into an RNA-induced silencing complex, siRNAs mediate cleavage of the homologous endogenous mRNA transcript by guiding the complex to the homologous mRNA transcript, which is then cleaved by the complex. The siRNA may be homologous to any region of the MTTGS transcript. The region of homology may be 30 nucleotides or less in length, preferably less than 25 nucleotides, more preferably about 21 to 23 nucleotides, most preferably about 19 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003). Typically, a target site that begins with AA, has 3′ UU overhangs for both the sense and antisense siRNA strands, and has an approximate 50% G/C content. siRNAs may be chemically synthesized, created by in vitro transcription, or expressed from an siRNA expression vector or a PCR expression cassette. See, e.g., website address ambion.com/techlib/tb/tb.sub.—506 (last retrieved May 10, 2006). Chemically synthesized siRNA relies on the same solid-phase support chemistry used to generate DNA primers for PCR. Expression or viral vectors and their RNA polymerase III (Pol III) promoters drive the expression of either siRNA transcripts, as separate sense and antisense strands that anneal in the cell, or a single short hairpin RNA transcript (Paddison, P. J. et al. (2002) Genes Dev. 16, 948-958; Sui, G. et al. (2002) Proc. Natl. Acad. Sci. U.S.A. 99, 6047-6052; Paul, C. P. et al. (2002) Nat. Biotechnol. 20, 505-508; Miyagishi M, et al. (2002) Nat. Biotechno1.20, 497-500). Human and mouse U6 and the human H1 are the most commonly used RNA polymerase III promoters. The polymerase III enzyme initiates and terminates RNA transcripts at well-defined positions (Goomer R S, et al. (1992) Nucleic Acids Res. September 25; 20(18):4903-12) making its promoters well suited for the synthesis of siRNA or shRNA.

An antisense inhibitory nucleic acid may also be used to specifically reduce MTTGS expression, for example, by inhibiting transcription and/or translation. An antisense inhibitory nucleic acid is complementary to a sense nucleic acid encoding MTTGS. For example, it may be complementary to the coding strand of a double-stranded cDNA molecule or complementary to an mRNA sequence. It may be complementary to an entire coding strand or to only a portion thereof. It may also be complementary to all or part of the noncoding region of a nucleic acid encoding MTTGS. The non-coding region includes the 5′ and 3′ regions that flank the coding region, for example, the 5′ and 3′ untranslated sequences. An antisense inhibitory nucleic acid is generally at least six nucleotides in length, but may be about 8, 12, 15, 20, 25, 30, 35, 40, 45, or 50 nucleotides long. Longer inhibitory nucleic acids may also be used.

An antisense inhibitory nucleic acid may be prepared using methods known in the art, for example, by expression from an expression vector encoding the antisense inhibitory nucleic acid or from an expression cassette. Alternatively, it may be prepared by chemical synthesis using naturally-occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the antisense inhibitory nucleic acid and the sense nucleic acid.

Naturally-occurring nucleotides include the ribose or deoxyribose nucleotides adenosine, guanine, cytosine, thymine and uracil. Examples of modified nucleotides include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, butoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

An inhibitor of the invention can also be a small hairpin RNA or short hairpin RNA (shRNA) is a sequence of RNA that makes a tight hairpin turn that can be used to silence gene expression via RNA interference. The shRNA hairpin structure is cleaved by the cellular machinery into an siRNA, which is then binds to and cleaves the target mRNA. shRNA can be introduced into cells via a vector encoding the shRNA, where the shRNA coding region is operably linked to a promoter. The selected promoter permits expression of the shRNA. For example, the promoter can be a U6 promoter, which is useful for continuous expression of the shRNA. The vector can, for example, be passed on to daughter cells, allowing the gene silencing to be inherited. See, McIntyre G, Fanning G, Design and cloning strategies for constructing shRNA expression vectors, BMC BIOTECHNOL. 6:1 (2006); Paddison et al., Short hairpin RNAs (shRNAs) induce sequence-specific silencing in mammalian cells, GENES DEV. 16 (8): 948-58 (2002).

An inhibitor of the invention may also be a ribozyme. A ribozyme is an RNA molecule with catalytic activity and is capable of cleaving a single-stranded nucleic acid such as an mRNA that has a homologous region. See, for example, Cech, Science 236: 1532-1539 (1987); Cech, Ann. Rev. Biochem. 59:543-568 (1990); Cech, Curr. Opin. Struct. Biol. 2: 605-609 (1992); Couture and Stinchcomb, Trends Genet. 12: 510-515 (1996). A ribozyme may be used to catalytically cleave a MTTGS mRNA transcript and thereby inhibit translation of the mRNA. See, for example, Haseloff et al., U.S. Pat. No. 5,641,673.

Methods of designing and constructing a ribozyme that can cleave an RNA molecule in trans in a highly sequence specific manner have been developed and described in the art. See, for example, Haseloff et al., Nature 334:585-591 (1988). A ribozyme may be targeted to a specific RNA by engineering a discrete “hybridization” region into the ribozyme. The hybridization region contains a sequence complementary to the target RNA that enables the ribozyme to specifically hybridize with the target. See, for example, Gerlach et al., EP 321,201. The target sequence may be a segment of about 5, 6, 7, 8, 9, 10, 12, 15, 20, or 50 contiguous nucleotides selected from a specific nucleotide sequence. Longer complementary sequences may be used to increase the affinity of the hybridization sequence for the target.

The hybridizing and cleavage regions of the ribozyme can be integrally related; thus, upon hybridizing to the target RNA through the complementary regions, the catalytic region of the ribozyme can cleave the target. Thus, an existing ribozyme may be modified to target a MTTGS nucleic acid of the invention by modifying the hybridization region of the ribozyme to include a sequence that is complementary to the target MTTGS nucleic acid. Alternatively, an mRNA encoding a MTTGS may be used to select a catalytic RNA having a specific ribonuclease activity from a pool of RNA molecules. See, for example, Bartel & Szostak, Science 261:1411-1418 (1993).

Thus, inhibitory nucleic acids of the invention may include modified nucleotides, as well as natural nucleotides such as combinations of ribose and deoxyribose nucleotides, and an antisense inhibitory nucleic acid of the invention may be of any length discussed above and that is complementary to MTTGS.

In some embodiments, expression cassettes are employed in the various embodiments described herein. Expression cassettes can be of any suitable construction, and can be included in any appropriate delivery vector. Such delivery vectors include plasmid DNA, viral DNA, and the like. The means by which the expression cassette in its delivery or expression vector is introduced into target cells or target organism can be transfection, reverse transfection, virus induced transfection, electroporation, direct introduction by biolystics (e.g., using a “gene gun;” BioRad, Inc., Emeryville, Calif.), and the like. Other methods that can be employed include methods widely known in the art as the methods of gene therapy. Once delivered into a target cell, or target organism the expression cassette may be maintained on an autonomously replicating piece of DNA (e.g., an expression vector), or may be integrated into the genome of the target cell or target organism.

Typically, to assemble the expression cassettes and vectors of the present invention a nucleic acid, preferably a DNA, encoding an siRNA is incorporated into a unique restriction endonuclease cleavage site, or a multiple cloning site, within a pre-existing “empty” expression cassette to form a complete recombinant expression cassette that is capable of directing the production of the siRNA transcripts of the present invention. Frequently such complete recombinant expression cassettes reside within, or inserted into, expression vectors designed for the expression of such siRNA transcripts. Methods for the construction of an expression vector for purposes of this invention should be apparent to skilled artisans apprised of the present invention. (See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.)

Generally, the expression cassettes inserted or assembled within the expression vectors have a promoter operably linked to a DNA encoding the siRNA that is to be employed. The promoter can be a native promoter, i.e., a promoter that is responsible for the expression of that particular gene product in cells, or it can be any other suitable promoter. Alternatively, the expression cassette can be a chimera, i.e., having a heterologous promoter that is not the native promoter responsible for the expression of the siRNA. Such heterologous promoters can even be from a different species than the target cell or organism.

The expression vector may further include an origin of DNA replication for the replication of the vectors in target cells. Preferably, the expression vectors also include a replication origin for the amplification of the vectors in, e.g., E. coli, and selection marker(s) for selecting and maintaining only those target cells harboring the expression vectors. Additionally, in some embodiments the expression vectors also contain inducible or derepressible promoters, which function to control the transcription of the siRNA transcript from the DNA that encodes it. Other regulatory sequences such as transcriptional enhancer sequences and translation regulation sequences (e.g., Shine-Dalgarno sequence) can also be operably included in the expression vectors. Transcription termination sequences, and polyadenylation signal sequences, such as those from bovine growth hormone, SV40, lacZ and AcMNPV polyhedral protein genes, may also be present.

The expression vectors of the present invention can be introduced into the target cells by any techniques known in the art, e.g., by direct DNA transformation, microinjection, electroporation, viral infection, lipofection, biolystics, and the like. The expression of the siRNA can be transient or stable, inducible or derepressible. The expression vectors can be maintained in target cells in an extrachromosomal state, i.e., as self-replicating plasmids or viruses. Alternatively, the expression vectors, or portions thereof, can be integrated into chromosomes of the target cells by conventional techniques such as site-specific recombination or selection of stable cell lines. In stable cell lines, at least the expression cassette portion of the expression vector is integrated into a chromosome of the target cells.

Another inhibitor embodiment may be antibodies specific to MTTGS polypeptide(s). As described above, one example of a therapeutic agent may pertain to an antibody. Any type of antibody known in the art can be generated to bind specifically to an epitope of an MUGS polypeptide. “Antibody” as used herein includes intact immunoglobulin molecules, as well as fragments thereof, such as Fab, F(ab′)₂, and Fv, which are capable of binding an epitope of an MTTGS polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of an MTTGS polypeptide can be used therapeutically, as mentioned, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen. Antibodies useful for embodiments of the subject invention may be polyclonal, but are preferably monoclonal antibodies.

### REFERENCES

1. Kalscheuer R, Steinbüchel A. (2003) A novel bifunctional wax ester synthase/acyl-CoA:diacylglycerol acyltransferase mediates wax ester and triacylglycerol biosynthesis in Acinetobacter calcoaceticus ADP1. J Biol. Chem. 278:8075-8082
2. Yen C L, Monetti M, Burri B J, Farese R V Jr. (2005) The triacylglycerol synthesis enzyme DGAT1 also catalyzes the synthesis of diacylglycerols, waxes, and retinyl esters. J Lipid Res. 46:1502-1511.
3. Smith S J, Cases S, Jensen D R, Chen H C, Sande E, Tow B, Sanan D A, Raber J, Eckel R H, Farese R V Jr. (2000) Obesity resistance and multiple mechanisms of triglyceride synthesis in mice lacking Dgat. Nat. Genet. 25:87-90.
4. Stone S J, Myers H M, Watkins S M, Brown B E, Feingold K R, Elias P M, Farese R V Jr. (2004) Lipopenia and skin barrier abnormalities in DGAT2-deficient mice. J Biol. Chem. 279:11767-11776.

TABLE 1
Mycobacterium tuberculosis TGS 1 (Rv3130c) is a dual-function
triacylglycerol synthase/wax synthase.

(nmol/mg/min)

| | TGS Activity | WS Activity |
|---|---|---|
| | ¹⁴C-Diolein + | ¹⁴C-C18:1-CoA + |
| | Oleoyl-CoA | Oleoyl alcohol |
| Rv3130c (MTTGS 1) | 3.91 | 1.13 |
| Rv3734c (MTTGS 2) | 0.46 | 0.02 |
| Rv3740c | 0.02 | 0.02 |

Triacylglycerol synthase (TGS) genes were expressed in E. coli and total cell lysates were assayed for TGS and wax synthase (WS) activities. The TGS assays were performed with lysates containing 150 μg protein in 0.1M citrate/phosphate buffer, pH 6.5, containing 1 mg BSA, 10 mM MgCl₂, 500 μM [¹⁴C]diolein (specific activity 1.6 Ci mol⁻¹), 100 μM oleoyl (C_{18:1})-CoA in a total volume of 250 μl for 2 h at 37° C. The WS assays contained 50 μM [¹⁴C]oleoyl-CoA (16 Ci mol⁻¹) and 100 μM oleyl(C_{18:1}) alcohol as substrates. Following the assay, the reaction mixtures were extracted and the TG or WS product was quantified as described previously (Daniel et al., 2004).
nd, not determined.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

The invention claimed is:

1. A method of screening for agents potentially useful in the treatment of Mycobacterium tuberculosis infection in a mammal comprising the steps of i) determining the wax synthase activity of a MTTGS polypeptide sequence in the presence of a test compound or in the absence of said test compound, and ii) identifying a test compound whose presence modulates the wax synthase activity of said MTTGS polypeptide as a potential therapeutic agent for decreasing the activity of Mycobacterium tuberculosis triacylglycerol synthase.

2. A method of screening for agents potentially useful in the treatment of Mycobacterium tuberculosis infection in a mammal comprising the steps of i) determining the wax synthase and triacylglycerol synthase activity of a MTTGS polypeptide sequence in the presence of a test compound or in the absence of said test compound, and (ii) identifying a test compound whose presence modulates the wax synthase activity more than the triacylglycerol synthase activity of said MTTGS polypeptide as a potential therapeutic agent for decreasing the activity of Mycobacterium tuberculosis triacylglycerol synthase.

3. The method of claim 2, wherein the test compound binds to the acyl alcohol binding pocket of MTTGS.

4. The method of claim 1, wherein said test compound contacts said MTTGS polypeptide.

5. The method of claim 4, wherein the step of contacting in or at the surface of a cell.

6. The method of claim 5, wherein the cell is in vitro.

7. The method of claim 4, wherein the step of contacting is in a cell-free system.

8. The method of claim 2, wherein the polypeptide is coupled to a detectable label.

9. The method of claim 2, wherein the compound is coupled to a detectable label.

10. The method of claim 2, wherein a ligand is bound to said polypeptide and the test compound displaces said ligand.

10. The method of claim 2, wherein the polypeptide is attached to a solid support.

11. The method of claim 2, wherein the compound is attached to a solid support.

12. A modulating agent identified by the method of claim 1.

13. A modulating agent identified by the method of claim 2.

13. A MTTGS inhibitor that selectively inhibits MTTGS wax synthase activity more than TG synthase activity.

14. An inhibitor of claim 13, comprising an acyl alcohol mimic.


SEQUENCE LISTING (3 sequences)

SEQ ID NO: 1 - DNA, 1392 bp, Mycobacterium tuberculosis
atgaatcacctaacgacacttgacgccgggtttctcaaggcagaagacgtggatcggcacgtgagtctggcaatcggcgctctggcggtcatcgaggggccggctcccgatcaggaagccttcttatcgtcgctcgctcaacgcctacgtccctgtacccggttcgggcagcggttacgcctgcgcccgttcgacctcggtgcacccaaatgggtggacgatcccgacttcgatcttggccgtcatgtgtggcgcatcgccttgccgcggcctggcaacgaagaccagttattcgagctgatcgccgatctgatggcgcgtcgtttggaccggggtcgaccgctgtgggaggtctgggtcatcgaaggcctggcggacagcaagtgggcgatcctgaccaaactgcaccactgcatggccgacggaatcgcggcgactcacctgctagctgggctctccgatgaaagtatgagcgacagcttcgcgagcaacatccacacgaccatgcagtcgcaatccgcatctgtgcggcggggtggattccgtgtcaatccaagcgaggcgttgaccgcgtcgaccgccgtgatggcaggcatcgttcgcgcggccaagggtgccagtgagatcgcggccggcgtgctaagtcccgccgcgtcgtcgttgaacgggccgatcagtgatttgcgtcgctacagcgcagcaaaggtccctctcgccgacgtcgaacaggtgtgccggaaattcgacgtcaccatcaatgatgttgcgcttgccgcgattacggaaagctaccgcaacgtcctcatccagcggggtgagcggcctaggtttgattcgctgcgtacgctagtgccggtctcgacgcgttccaacagcgctttgagcaagaccgataaccgtgtttcgttaatgctgcccaacctgccggtggatcaagagaacccgctgcagcggctgcggatcgtgcactcgcggctgactcgggccaaggcggggggacagagacaattcggaaatactttgatggcgattgccaaccgccttccgttccccatgaccgcatgggcggtcgggctgttgatgcggctgccgcagcgtggtgttgtcaccgtggcgacaaatgtgccgggtccacgacggccgctgcagattatgggcagacgggtgcttgacctatacccggtttcgccgatcgcgatgcaactgcgcaccagtgtcgcgatgctcagctacgccgacgacctgtacttcgggatcctggccgactacgacgtggtagcagatgccggccagctggcgcgaggaattgaagacgccgtcgcacggctggtggcgatcagtaagcggcgcaaggtgactcgcaggcgcggagcgctatcgctggttgtgtga

SEQ ID NO: 2 - protein, 463 aa, Mycobacterium tuberculosis
MNHLTTLDAGFLKAEDVDRHVSLAIGALAVIEGPAPDQEAFLSSLAQRLRPCTRFGQRLRLRPFDLGAPKWVDDPDFDLGRHVWRIALPRPGNEDQLFELIADLMARRLDRGRPLWEVWVIEGLADSKWAILTKLHHCMADGIAATHLLAGLSDESMSDSFASNIHTTMQSQSASVRRGGFRVNPSEALTASTAVMAGIVRAAKGASEIAAGVLSPAASSLNGPISDLRRYSAAKVPLADVEQVCRKFDVTINDVALAAITESYRNVLIQRGERPRFDSLRTLVPVSTRSNSALSKTDNRVSLMLPNLPVDQENPLQRLRIVHSRLTRAKAGGQRQFGNTLMAIANRLPFPMTAWAVGLLMRLPQRGVVTVATNVPGPRRPLQIMGRRVLDLYPVSPIAMQLRTSVAMLSYADDLYFGILADYDVVADAGQLARGIEDAVARLVAISKRRKVTRRRGALSLVV

SEQ ID NO: 3 - protein, 7 aa, Mycobacterium tuberculosis
HHCMADG